(19) United States
(12) Patent Application Publication
(10) Pub. No.: US 2016/0220604 A1
(43) Pub. Date: Aug. 4, 2016

(54) MAGNESIUM-LIPOSOME COMPLEXES

(71) Applicant: FRESENIUS MEDICAL CARE DEUTSCHLAND GMBH, Bad Homburg (DE)

(72) Inventors: Janine BUECHEL, Bad Homburg (DE); Wolfgang HOFMANN, Frankfurt am Main (DE); Kristina GUNDLACH, Mainz (DE); Jasmin YILLAH, Frankfurt am Main (DE); Sonja STEPPAN, Neu-Isenburg (DE); Hans-Juergen ARENS, Oberursel (DE)

(73) Assignee: Fresenius Medical Care Deutschland GmbH, Bad Homburg (DE)

(21) Appl. No.: 15/022,268

(22) PCT Filed: Sep. 16, 2014
(86) PCT No.: PCT/EP2014/069729

§ 371 Date: Mar. 16, 2016

(30) Foreign Application Priority Data
Sep. 17, 2013 (DE) 10 2013 015 334.4

(51) Int. Cl. A61K 33/06; A61K 9/127

(52) U.S. Cl. CPC A61K 33/06; A61K 9/1271; A61K 9/1277

(57) ABSTRACT

The present invention relates to magnesium-liposome complexes with a delayed release of magnesium, coated at least partially with a polyalkylene glycol compound. In addition, the present invention relates to methods for synthesis of the magnesium-liposome complexes as well as use thereof in the treatment of diseases associated with a magnesium deficiency.

The present invention relates to magnesium-liposome complexes with a delayed release of magnesium and methods for producing same. In addition, the present invention relates to magnesium-liposome complexes for use in the treatment of diseases associated with a magnesium deficiency.

Magnesium is the fourth-most common cation in the human body and acts as a coenzyme with more than 300 enzymes. Magnesium is important for the formation of ATP and nucleic acid as well as for muscle function.

Despite its importance, many patients unknowingly have a magnesium deficiency, i.e., are hypomagnesemic, which means that their magnesium levels are too low.

This may have clinical effects because hypomagnesemia is associated with diabetes mellitus, dyslipidemia, endothelial dysfunction, oxidative stress, asthma, hypertension and inflammation. Inflammation and calcification of the blood vessels in particular lead to cardiovascular problems and may cause of premature death. Pathological cardiovascular changes are widespread and are associated with a number of other diseases. For example, they are the most common cause of death in patients with chronic kidney disease (CKD).

It has been shown that higher magnesium levels in CKD patients are associated with reduced calcification and a lower mortality. In addition, magnesium inhibits unwanted calcification through a direct cellular effect.

The required daily dose of approximately 300 mg is usually achieved through a balanced diet of magnesium-rich foods such as whole grain products, edible nuts, fish or mineral water. An increased demand can be met by nutritional supplements or medication. This may be accomplished by tablets, in particular effervescent tablets, for example.

However, in CKD patients there is the problem of compliance with oral administration of magnesium. Effective monitoring of intake is impossible because the tablets are taken at home without medical supervision. Patients with kidney disease must take at least 12 tablets a day, such as iron preparations, phosphate and potassium binders. A daily regimen of up to 30 tablets is not unusual. Meanwhile these patients are allowed to drink a maximum of only one liter of water. Consequently, many tablets are not even taken.

In addition, oral administration of magnesium leads to gastrointestinal irregularities. The poorly absorbable magnesium ions lead to an osmotic gradient in the intestine, resulting in an influx of fluid into the digestive tract. This increases the fluidity of the intestinal content and consequently has a laxative effect.

Another option is to administer magnesium intravenously in the form of a sterile magnesium sulfate solution. In emergency medicine, this is used for treatment of eclampsia, for example.

Respiratory arrest is a feared adverse effect of intravenous administration of magnesium. It is usually manifested first by failure of autonomic reflexes (areflexia). Intravenous magnesium should therefore be administered only slowly. Other adverse effects include headaches, palpitations, dizziness, nausea and vomiting.

Intravenous administration of a magnesium sulfate solution leads to a marked short-term increase in serum magnesium levels, but they then drop again rapidly. A bolus of magnesium sulfate is not suitable for elevating the magnesium level in the human body over a longer period of time, for example, for hours or days.

It is known from the prior art that drugs may be embedded in liposomes. Liposomal formulation of drug may be used to make lipophilic active ingredients bioavailable. Furthermore, when sensitive drugs are incorporated into liposomes, they can be protected from possibly being metabolized after administration to thereby increase the plasma half-life of the active ingredient.

Liposomes as a “drug delivery system” permit a targeted and selective transport of drugs to the locations in the body where they are needed. Adverse effects of the lipsomally formulated medication are therefore reduced, and since lower doses can be administered, the efficacy and therapeutic scope are increased. Hydrophilic drugs are enclosed and are situated in the hydrophilic interior of the liposome. Lipophilic drugs are embedded in the membrane, and amphiphilic drugs are located at the interface between the interior of the membrane and the aqueous phase.

Magnesium-liposome complexes are known from the prior art. U.S. Pat. No. 5,501,859 discloses an absorbable magnesium composition for oral administration, comprising a magnesium salt and liposomes of 1-palmitoyl-2-oleoyl-phosphatidylcholine.

WO 88/07852 discloses positively charged unilamellar liposomes containing magnesium for intravenous, intramuscular or subcutaneous administration. However, positively charged liposomes are toxic and are rapidly detected by the mononuclear phagocyte system and removed from the bloodstream. Positively charged liposomes are thus also unsuitable for elevating the magnesium level in the human body over a longer period of time, for example, hours or days.

### DESCRIPTION OF THE INVENTION

The present invention relates to magnesium-liposome complexes with delayed release of magnesium for intravenous administration.

The present relates in particular to magnesium-liposome complexes coated with a polyalkylene glycol compound.

The magnesium salt incorporated into the liposomes comprises any pharmaceutically acceptable magnesium salt obtainable from the reaction of magnesium with an acid selected from hydrochloric acid, hydrobromic acid, phosphoric acid, nitric acid, sulfuric acid, acetic acid, 2,2-dichloroacetic acid, adipic acid, ascorbic acid (D- or L-form thereof, in particular the L-form thereof), aspartic acid (D- or L-form thereof, in particular the L-form thereof), benzenesulfonic acid, benzoic acid, 4-acetamidobenzoic acid, camphoric acid ((+)-form or (−)-form thereof, in particular the (+)-form thereof), camphor-10-sulfonic acid ((+)-form or (−)-form thereof, in particular the (+)-form thereof), capric acid (decanoic acid), caproic acid (hexanoic acid), caprylic acid (octanoic acid), carbonic acid, cinnamic acid, citric acid, cvclamic acid, dodecyl-sulfuric acid ester, ethane-1,2-disulfonic acid, ethanesulfonic acid, 2-hydroxyethanesulfonic acid, formic acid, fumaric acid, calactaric acid, gentisic acid, glucoheptonic acid (D- or L-form thereof, in particular the D form thereof), gluconic acid (D- or L-form thereof, in particular the D-form thereof), glucuonic acid (D- or L-form thereof, in particular the D-form thereof), glutamic acid, giutaric acid, 2-oxoglutaric acid, cavcerophosphoric acid, glycolic acid, hippuric acid, isobutyric acid, lactic acid (D- or L-form thereof), lactobionic acid, lauric acid, maleic acid, malic acid (D- or L-form thereof), maionic acid, mandelic acid (D- or L-form thereof), methanesulfonic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid (embonic acid), propionic acid, pyrogiutamic acid (D- or L-form thereof, in particular the L-form thereof), salicylic acid, 4-aminosalicylic acid, sebacic acid, stearic acid, succinic acid, tartaric acid (D- or L-form thereof), thiocyanic acid, toluenesulfonic acid (in particular the p-isomer) and undecylenic acid or their solvates, hydrates and mixtures thereof.

The magnesium salt is preferably water-soluble. Magnesium sulfate and magnesium chloride are preferred in particular.

The magnesium-liposome complexes are preferably unilameliar, comprising approximately 50% phosphatidylcholine, approximately 25-30% phosphatidylethanolamine, approximately, 10% phosphatidylserine, and 10% additional ingredients. The particularly preferred fatty acids are oleic acid, palmitic acid, and linolenic acid.

Unilamellar liposomes can be synthesized by gradual dropwise addition of ethanolic or ether-containing lipid solutions into the aqueous phase, by extrusion of multilamellar vesicles (MLV) through membranes of a certain pore size (for example, 100 nm), by dialysis of detergent-lipid mixtures or by ultrasonic treatment and vibration of aqueous solutions on previously dried lipid films.

The charge of the magnesium-liposome complexes according to the invention may be positive, negative or neutral. In a preferred embodiment, the charge of the magnesium-liposome complexes according to the invention is neutral. It is known from the prior art that positively charged liposomes are toxic and therefore are detected rapidly by the mononuclear phagocyte system and removed from the bloodstream. In addition, negatively charged liposomes bind more strongly than neutral liposomes to cells and are absorbed into the cells by endocytosis to a greater extent. It has been found that neutral magnesium-liposome complexes cause less complement activation than positively or negatively charged liposomes and therefore have a longer half-life in the bloodstream.

The magnesium-liposome complexes according to the invention have an average diameter of less than 400 nm, preferably 70 to 250 nm. An average diameter of 100 to 200 nm is especially preferred because magnesium-liposome complexes of this size are degraded in the liver or by macrophages only with a time lag.

The size distribution of the vesicles of less than approximately 1000 nm can be determined with the help of dynamic or static light scattering, gel permeation chromatography or microscopy.

The magnesium-liposome complexes according to the invention are characterized in that the liposome is modified with a compound comprising a polyalkylene glycol chain. The polyalkylene glycol may be, for example, polyethylene glycol (PEG), polypropylene glycol or the like.

In a preferred embodiment, the polyalkylene glycol is polyethylene glycol with a molecular weight of approximately 500-7000 Dalton, preferably 1000-5000 Dalton.

The surface modification of liposomes with polyalkylene glycol. such as polyethylene glycol, for example, may be accomplished in various ways. One possibility is to physically adsorb the polyethylene glycol on the surface of liposomes. However, polyethylene glycol is preferably anchored on the surface of liposomes by means of covalent chemical bonding. Two synthesis routes are available for this purpose. First, polyethylene glycol may be anchored on finished liposomes. The lipids incorporated into the liposome have at least one reactive group, such as maleimide groups, for example, to which the polyethylene glycol chain is covalently bound. Second, there is the option of synthesizing polyethylene glycol-lipid conjugates in a first step and then using them in the synthesis of the liposomes in a second step. Poly (ethylene glycol)-phosphatidylethanolamine (PEG-PE) is one example of such a polyethylene glycol-lipid conjugate.

The amount of polyalkylene glycol compound used to coat the magnesium-liposome complexes is not particularly limited. A preferred amount of polyalkylene glycol to be added is approximately 0.2 to 5 mol %, preferably approximately 0.28 to 3 mol %, especially preferably 0.3 to 2.5 mol %, based on the total lipids.

The present invention also relates to pharmaceutical formulations suitable for intravenous administration of the magnesium-liposome complexes according to the invention. In a preferred embodiment of the present invention, the magnesium-liposome complexes according to the invention are lyophilized. Pharmaceutical formulations containing the magnesium-liposome complexes according to the invention may also include additional excipients such as disaccharides, in particular sucrose, lactose or trehalose.

The lyophilized magnesium-liposome complexes may be packaged in ampoules, vials or injection vials. Before they are administered, a suitable suspension medium, such as isotonic saline solution, is added and a liposome suspension is prepared.

The present invention also relates to a dosing regimen for administering magnesium-liposome complexes, such that this dosing regimen delays the degradation of the magnesiumliposome complexes by the mononuclear phagocyte system.

The degradation can be delayed by administering “empty” liposomes in advance, i.e., liposomes without any magnesium salt, and saturating the mononuclear phagocyte system in this way. Then the magnesium-containing liposomes are administered.

In another embodiment, “empty” liposomes and those containing magnesium are administered concurrently. The substance quantity ratio of the “empty” liposomes to the liposomes containing magnesium is 10:1 to 1:10, preferably 5:1 to 1:2, especially preferably 2:1 to 1:1.

Another preferred embodiment relates to pharmaceutical formulation containing magnesium-free and magnesium-containing liposomes in a substance quantity ratio of 10:1 to 1:10, preferably 5:1 to 1:2, especially preferably 2:1 to 1:1.

The magnesium-liposome complexes according to the invention are suitable for use in the prevention and treatment of diseases associated with hypomagnesemla.

The magnesium-liposome complexes according to the invention are suitable for use in the treatment of patients with an increased risk of cardiovascular diseases, in particular CKD patients, regardless of their serum magnesium levels.

The magnesium-liposome complexes according to the invention are suitable in particular for use in the prevention and treatment of inflammatory vascular diseases.

The magnesium-liposome complexes according to the invention are suitable in particular for use in the prevention and treatment of vascular calcification.

The magnesium-liposome complexes according to the invention are suitable for use in the treatment of patients with metabolic syndrome, type 2 diabetes mellitus (non-insulin-dependent diabetes mellitus (NIDDM)). The magnesium-liposome complexes according to the invention are suitable for use in the prevention and treatment of preeclampsia or eclampsia.

The magnesium-liposome complexes according to the invention are suitable for use in he prevention and treatment of elevated blood pressure.

The magnesium-liposome complexes according to the invention are suitable for use in the prevention and treatment of fatigue syndrome.

### EXAMPLES

The present invention is explained with reference to the examples.

### Example 1: Preparation of a Liposome and Encapsulation of a Magnesium Salt

A lipid mixture (3.2 g) containing dipaimitoyl-phosphatidylcholine (DFPC), cholesterol and dipalmitoyl-phosphatidyl-ethanolamine (DFPE) in a molar ratio of 18:10:0.5 is placed in 32 mL 0.3M citrate buffer (pH 4.0) and hydrated. Multilamellar liposomes are then produced by freezing and thawing the mixture three times using liquid nitrogen and a heating bath (60° C.) The suspensions are extruded several times through isoporic polycarbonate membranes with a pore size of 200 nm. The resulting liposome solution is neutralized by dropwise addition of 1M NaCH. Then the solution is heated to 60° C. and an aqueous magnesium chloride solution is added in an amount of 0.5 mL of 20 mg/mL per 100 mg lipids for the encapsulation.

### Example 2: Synthesis of a PEG Protected Magnesium-Liposome Complex of Poly-(Ethylene Glycol)-Phosphatidylethanolamine (PEG-PE)

A lipid mixture (3.2 g) containing dipalmitoyl-phosphatidylcholine (DPEC), cholesterol and poly-(ethylene glycol)-phosphatidylethanolamine (PEG-PE) in a molar ratio of 13:10:0.5 is placed in 32 mL 0.3M citrate buffer (pH 4.0) and hydrated. Multilamellar liposomes are then produced by freezing and thawing the mixture three times using liquid nitrogen and a heating bath (60° C.). The suspensions are extruded several times through isoporic polycarbonate membranes with a pore size of 200 nm. The resulting liposome solution is neutralized by dropwise addition of 1M NaOH. Then the solution is heated to 60° C. and an aqueous magnesium chloride solution is added in an amount of 0.5 mL of 20 mg/mL per 100 mg lipids for the encapsulation.

1. A manesium-liposome complex,
characterized in that
the liposomes are coated at least in part with a polyalkylene glycol compound.

2. The maqnesium-liposome complex according to claim 1,
wherein the polyalkylene glycol compound is polyethylene glycol.

3. The magnesium-liposome complex according to claim 1,
wherein the average molecular weight of the polyethylene glycol radicals is 500 to 7000 Dalton, preferably 1000 to 5000 Dalton.

4. The magnesium-liposome complex according to claim 1,
wherein the average liposome size is 70 to 250 nm, preferably 100 to 250 nm, especially preferably 100 to 200 nm.

5. The magnesium-liposome complexes according to claim 1,
wherein the complex comprises a magnesium salt obtainable from the reaction of magnesium with an acid selected from the reaction of magnesium with an acid selected from hvdrochloric acid, hydrobromic acid, phosphoric acid, nitric acid, sulfuric acid, acetic acid, 2,2-dichloroacetic acid, adipic acid, ascorbic acid (D- or L-form thereof, in particular the L-form thereof), aspartic acid (D- or L-form thereof, in particular the L-form thereof), benzenesulfonic acid, benzoic acid, 4-acetamido-benzoic acid, camphoric acid ((+) form or (−) form thereof, in particular the (+)-form thereof), camphor-10-sulfonic acid ((+) form or (−) form thereof, in particular the (+)-form thereof), capric acid (decanoic acid), caproic acid (hexanoic acid), caprylic acid (octanoic acid), carbonic acid, cinnamic acid, citric acid, cyclamic acid, dodecylsulfuric acid ester, ethane-1,2-disulfonic acid, ethanesulfonic acid, 2-hydroxy ethanesulfonic acid, formic acid, fumaric acid, galactaric acid, gentisic acid, glucoheptonic acid, (D- or L-form thereof, in particular the D-form thereof) gluconic acid (D- or L-form thereof, in particular the D-form thereof) glucuronic acid (D- or L-form thereof, in particular the D-form thereof), glutamic acid, qiutaric acid, 2-oxoglutaric acid, glycerophosporic acid, glycolic acid, hippuric acid, isobutyric acid, lactic acid (D- or L-form thereof), lactobionic acid, lauric acid, maleic acid, malic acid (D- or L-form thereof), malonic acid, mandelic acid (D- or L-form thereof), methanesulfonic acid, naphthalene 1,5-disulfonic acid, naphthalene-2-sulfonic acid, 1-hydroxy-2-naphthoic acid, nicotinic acid, oleic acid, orotic acid, oxalic acid, palmitic acid, pamoic acid (embonic acid) propionic acid, pyroglutamic acid (D- or L-form thereof, in particular the L-form thereof), salicylic acid, 4-aminosalicylic acid, sebacic acid, stearic acid, succinic acid, tartaric acid (D- or L-form thereof), thiocyanic acid, toluenesulfonic acid (in particular the p isomer) and undecyienic acid or their solvates, hydrates and mixtures thereof.

6. A pharmaceutical composition containing magnesium-liposome complexes according to claim 1.

7. The pharmaceutical composition according to claim 6, wherein the magnesium-liposome complexes are lyophilized.

8. The pharmaceutical composition according to claim 6,
wherein the composition additionally contains magnesium-free liposomes.

9. A method for synthesis of a magnesium-liposome complex according to claim 1,
comprising the steps:
a. preparation of a lipid mixture
b. extrusion of the lipid mixture
c. encapsulation of a magnesium salt.

10. A magnesium-liposome complex according to claim 1,
for use in the treatment of a disease associated with hypomagnesemia.

11. The magnesium-liposome complex according to claim 1,
for use in the treatment of cardiovascular diseases, in particular in CKD patients.

12. The magnesium-liposome complex according to claim 1,
for use in the treatment of metabolic syndrome or type 2 diabetes mellitus (non-insulin-dependent diabetes mellitus (NIDDM)).

13. The magnesium -liposome complex according to claim 1,
for use in the prevention and treatment of vascular calcification.

14. The magnesium-liposome complex according to claim 1,
for use in the prevention and treatment of preeclampsia or eclampsia.

15. The magnesium-liposome complex accordine to claim 1,
for use in the prevention and treatment of elevated blood pressure.

16. The magnesium-liposome complex according to claim 1,
for use in the treatment of fatigue syndrome.
